(19) United States
(12) Patent Application Publication
(10) Pub. No.: US 2005/0165370 A1
(43) Pub. Date: Jul. 28, 2005

(54) Absorbent hygiene article provided with a wet indicator

(76) Inventors: Scott Smith, Greensboro, NC (US); Frank Markus, Tonisvorst (DE)

Correspondence Address: Smith Moore, P O Box 21927, Greensboro, NC, 27420, US

(21) Appl. No.: 10/498,851

(22) PCT Filed: Nov. 28, 2002
(86) PCT No.: PCT/EP02/13435

(30) Foreign Application Priority Data
Dec. 14, 2001 (DE) 101 61 495.0

(51) Int. Cl. A61F 13/15; A61F 13/20

(52) U.S. Cl. 604/361; 604/367

(57) ABSTRACT

The present invention relates to an absorbent hygiene article, comprising (a) a stiff element which stiffens on contact with body fluids, or (b) a gas element which forms a gas on contact with body fluids, or both as an indicator and (c) At least one hygiene article component, wherein the stiff element or the gas element which forms a gas or both are in contact with the hygiene article component.

### FIELD OF INVENTION

The invention relates to an absorbent hygiene article, a process for its production and the use of this hygiene article for reducing the tendency of a wearer to wetting themselves or as a wetting indicator.

### BACKGROUND OF INVENTION

Diapers are available in different sizes and with different absorption capacities depending on the age of the wearer, in particular of the baby. Owing to the great wearing comfort of diapers today and to their absorption capability, children are, as a rule, dry later than a few years ago. It is advantageous for the becoming dry of the child if the child can recognize that it has wet itself.

In this context, special diapers or diaper pants have been developed for small children, in which by means of various devices it is indicated to the child and the mother or both that the child has wet itself in the diaper and the diaper should therefore be changed.

The prior art proposes in this context various solutions. In WO 00/00232, the wetting is indicated by means of an electronic sensor. In WO 97/10789, an indicator is disclosed, which generates a physical signal for the wearer via capillary forces. U.S. Pat. No. 5,468,236 discloses an indicator, which generates a visual signal, which is generated by a chemical path.

These measures known from the previously mentioned prior art lead to both the child and the mother being able to notice the wetting substantially earlier than would be the case with common diapers without indicators of this type. The previously mentioned measures have however the disadvantage, that both mother and child receive the indication of the wetting at the same time. This does not lead to an increased independence of the child. Rather, a diaper change is generally carried out because of the perception of the mother. A greater incentive effect would be present, which would result in an earlier independence of the child, if the child of itself came to the mother with the desire to change the diaper.

WO 96/19168 discloses diapers, which comprise means with which the diaper wearer can feel a temperature change, dampness or a deformation in the diaper. The temperature change is enabled by means of certain substances, which react on contact with an aqueous liquid to release or absorb heat, while a sensation of dampness is achieved by special diaper layers, which retain moisture. A deformation of the diaper is achieved by working special materials, such as cellulose sponges, into the body of the diaper, which then swell on contact with an aqueous liquid. The disadvantage of these cellulose sponges exists, however, in their limited swelling capacity as well as in the high production costs of these materials.

The object according to the invention thus consists in general in overcoming the disadvantages arising from the prior art. In particular, an object according to the invention lies in making available a hygiene article with a wetting indicator, which indicates to the wearer, without the environment of the wearer noticing before the wearer, that the hygiene article has been wet. This indication should however, occur at least without substantial negative influence of the other performance features of the hygiene article. Furthermore, this wetting indicator should be based upon economical materials.

### SUMMARY OF INVENTION

The object according to the invention is solved by an absorbent hygiene article comprising
(a) a stiff element, which stiffens on contact with body liquids or
(b) a gas element, which forms a gas on contact with body fluids, or both as an indicator and
(c) at least one hygiene article component,
   whereby the stiff element or the gas element which forms a gas or both are in contact with the hygiene article component.

It is preferred according to the invention that the stiff element reduces the flexibility of the hygiene article. Preferably, the flexibility is so far reduced, that it becomes markedly more difficult for the wearer to move with the hygiene article. The flexibility should however, not be so far reduced that the wearer becomes mobile with difficulty or completely immobile.

In addition, it is preferred according to the invention that the stiff element is a resin, which stiffens on contact with body liquids or an inorganic compound, which stiffens on contact with body liquids.

Preferably, a gas, which is harmless to health, is formed by means of the gas element. It is further preferred that the gas element forms this gas in such quantities, that these are noticeable for the wearer and, just as with the reduction of flexibility, reduce the wearing comfort of the hygiene article, however do not lead to a painful sensation for the wearer.

By body, liquids are understood according to the invention serous fluids, blood and urine, preferably urine, which originates from the wearer of the hygiene article according to the invention.

### BRIEF DESCRIPTION OF THE DRAWINGS

FIG. 1 to 3 illustrate sections through the construction of a hygiene article.

FIGS. 4 and 5 show the view of a hygiene article construction.

FIG. 6 shows a cross section for a test for the determination of the stiffness of a diaper.

### DESCRIPTION OF THE PREFERRED EMBODIMENTS

According to the invention, a resin comprising polyurethane is preferred as stiffening resin, as for example described in U.S. Pat. No. 4,774,937, which is incorporated by reference herein. Preferred stiffening inorganic compounds according to the invention are plaster or systems comprising plaster. Among the systems comprising plaster those in particular are preferred which are used as dressing materials, for example for fixing broken bones in orthopaedic. The systems comprising plaster are preferably characterized in that plaster compounds are absorbed in a matrix. This matrix is preferably fiber-nonwovens (“Fasergelege”), fabrics or webs, wherein webs are particularly preferred. The proportion of plaster in the system comprising plaster preferably amounts to at least about 10 wt. %, particularly preferably at least about 30 wt % and even more preferably about 50 wt. %, based in the system. In a particular embodiment of the absorbent hygiene article according to the invention, this comprises a system comprising a plaster, wherein the system comprises plaster in a quantity in a range between about 35 and about 70 wt. %.

It is preferred according to the invention, that the gas element, which forms a gas, forms CO₂ or N₂, wherein the formation of CO₂ is preferred. In general, all known CO₂-forming chemical compounds known to the skilled person are considered, wherein among these carbonates and hydrogen carbonates of alkaline or alkaline earth metals are particularly preferred. Among these sodium hydrogencarbonate, potassium hydrogencarbonate, magnesium hydrogencarbonate, calcium hydrogencarbonate are preferred, and sodium hydrogencarbonate is particularly preferred. The above-named compounds are preferably used together with an acid in such a quantity that the small pH value on contact with this mixture of the CO₂-forming compound and the acid with an aqueous liquid lies in a range from about 4 to about 9, preferably from about 5 to about 8 and particularly preferably from about 6.5 to about 7.5.

As acid, in particular organic acids are preferred, wherein polybasic acids are particularly preferred. Among the organic acids formic acid, citric acid and acetic acid are preferred, whereby citric acid in consequence of its odour neutrality is particularly preferred. The gas element which forms a gas comprises preferably at least one of the above described compounds in a quantity of at least about 10 wt %, preferably at least about 30 wt % and particularly preferably at least about 50 wt. %, respectively based upon the gas element which forms a gas.

It is further preferred, according to the invention, that the stiff element or the gas element forming a gas or both are at least partially, preferably fully, surrounded by a layer, whereby from the design of this layer and the type and method of surrounding care is taken that no worsening of the wearing comfort or the hygiene articles or health risks by the use of the stiff element or the gas element which forms a gas arise. These layers further insure their provided position in the hygiene article of the stiff element or the gas element, which forms a gas. In this context, semi-permeable sheets are particularly suitable, which on the one hand enable the entry of body liquids, so that the stiff element or the gas element which forms a gas can come into contact with the body liquid, however on the other hand, prevent a too rapid escape of materials liberated by means of the stiff element or the gas element which forms a gas. In particular, with respect to the gas element, which forms a gas, it is advantageous, if these sheets retain the gas formed for so long, until the hygiene article has become at least partially inflated by means of the gas formed. This leads to a tighter fit of the hygiene article and signals the entry of body liquids into the hygiene article to the wearer by the altered wearing comfort. Layers and/or sheets of this type preferably comprise thermoplastic polymers and copolymers as well as thermoplastic elastomers, for example polyamides such as nylon, polyesters such as polyethylene terephthalate, polyolefins such as polyethylene or polypropylene, copolymers of ethylene and ethyl acrylate, thermoplastic polyurethanes, thermoplastic polyesters, wherein polypropylene, polyethylene and copolymers of polyethylene and polypropylene are particularly preferred.

Each of the two elements, preferably the gas element which forms a gas, can have, in addition to the substances causing the gas formation absorbent material different to these substances; this preferably in a quantity of at most about 10 wt %, particularly preferably at most about 40 wt % and more preferably of at most about 60 wt. %, respectively based on the respective element.

According to the invention it is further preferred that the hygiene article comprises as hygiene component (A) a cover layer which is at least partially permeable to body fluids, (B) optionally, preferably necessarily, an outer layer, (C) optionally, preferably necessarily, a distribution layer, (D) an absorption layer as well as (E) a layer comprising the stiff element or the gas element which forms a gas or both or preferably several of these elements, wherein the cover layer, optionally, preferably necessarily, the distribution layer as well as the absorption layer, are in at least partial contact with a liquid if the article is brought into contact with the liquid and optionally, preferably necessarily, are at least partially surrounded by the outer layer, whereby the stiff element or the gas element which forms a gas or both is arranged at least partially according to a variant (i) between the absorption layer and the outer layer, or (ii) between the distribution layer and the absorption layer or (iii) in the absorption layer or in at least two of the variants (i) to (iii). Particularly preferred according to the invention is a hygiene article, which comprises all hygiene article components (A) to (E).

As body fluid-permeable cover sheet, all materials, which allow liquid to pass through known to the skilled person are considered. It is preferred according to the invention that the cover layer is formed from a fibre-comprising material, whereby the fibres are preferably laid, woven or webbed, so that a surface structure with pores forms, whereby the pores are shaped in such a way that the body fluid can pass through the cover layer so quickly that upon wetting no body fluid puddles form on the cover layer.

In the hygiene article according to the invention, the outer layer is preferably impervious to fluids than the cover layer. Preferably, the outer layer is formed from polyethylene or polypropylene sheets, into which optionally fibres can be worked. The outer layer is constructed so that it is as good as impermeable to body fluids, but has a certain gas permeability, so that for example gases formed by the wearer of the hygiene article can escape through the outer layer. In this context it is preferred that the outer layer has a sufficient porosity, whereby the water vapour permeability (Water Vapour Transmission, ASTM E 96) lies from about 400 to about 6000 g/m² in 24 h, preferably from about 750 to about 2800 g/m² in 24 h, particularly preferably from about 1000 to about 2600 g/m² in 24 h.

In a hygiene article according to the invention, the distribution layer provides that the body fluid entering in a limited, in comparison to the total surface of the cover layer of the hygiene articles small, region, this distributed so quickly over the absorption layer arranged under the distribution layer that no congestion of the body fluid arises on the cover layer. In an embodiment of the hygiene article according to the invention the distribution layer and the absorption layer are combined together in such a way that a single layer is present which functions both as distribution layer and as absorption layer. Preferably, the distribution layer comprises artificial or cellulose fibers, which are laid, interlaced or webbed, preferably laid.

The absorption layer comprises an absorbent material. By absorbent material are understood materials, which can absorb at least about 10%, preferably at least about 50% and particularly preferably at least 100% of their own weight in water. In an absorption layer are contained preferably about 10 wt %, preferably at least about 50 wt % and particularly preferably at least about 70 wt. % of absorbent material, based on the absorption layer. It is particularly preferred that the absorption layer comprises as absorbent material an absorbent polymer.

The absorbent polymer is preferably based upon
(α1). from about 0.1 to about 99.999 wt. %, preferably from about 20 to about 98.99 wt. % and particularly preferably from about 30 to about 98.95 wt. % of polymerised, ethylenically unsaturated, acid group-containing monomers or salts thereof or polymerised, ethylenically unsaturated monomers containing a protonated or quarternated nitrogen, or mixtures thereof, wherein mixtures comprising at least ethylenically unsaturated, acid group-containing monomers, preferably acrylic acid are particularly preferred,
(α2) from 0 to about 70 wt. %, preferably from about 1 to about 60 wt. % and particularly preferably from about 1 to about 40 wt. % of polymerised, ethylenically unsaturated monomers which are co-polymerizable with (α1),
(α3) from 0 to about 10 wt. %, preferably from 0.01 to about 7 wt. % and particularly preferably from 0.05 to about 5 wt. % of one or more crosslinkers,
(α4) from 0 to about 30 wt. %, preferably from about 1 to about 20 wt. % and particularly preferably from about 5 to about 10 wt. % of water soluble polymers, and
(α5) from 0 to about 20 wt. %, preferably from about 0.01 to about 7 wt. % and particularly preferably from about 0.05 to about 5 wt. % of one or more auxiliaries, wherein the sum of the weight quantities (α1) to (α5) amounts to 100 wt. %.

The monoethylenically unsaturated, acid group-containing monomers (α1) can be partially or fully, preferably partially, neutralized. Preferably the monoethylenically unsaturated, acid group-containing monomers are neutralized to at least about 25 mol. %, particularly preferably to at least about 50 mol. % and further preferred from about 50 to about 90 mol. %. The neutralization of the monomers (α1) can occur before or also after polymerisation. Furthermore, the neutralization can occur with alkali metal hydroxides, alkaline earth metal hydroxides, ammonia as well as carbonates and bicarbonates. In addition, each further base is conceivable, which forms a water soluble salt with the acid. A mixed neutralization with different bases is also conceivable. Neutralization with ammonia or with alkali metal hydroxides is preferred, particularly preferred with sodium hydroxide or with ammonia.

Furthermore, the free acid groups can predominate in a polymer, so that this polymer has a small pH value lying in the acidic range. This acidic water absorbent polymer can be at least partially neutralized by means of a polymer with free basic groups, preferably amine groups, which is basic in comparison to the acidic polymer. These polymers are referred to in the literature as “Mixed-Bed Ion-Exchange Absorbent Polymers” (MBIEA-polymers) and are disclosed in WO 99/34843, among others. The disclosure of WO 99/34843 is hereby introduced as reference and thus forms part of the disclosure. As a rule, MBIEA-polymers represent a composition, which comprises on the one hand basic polymers, which can exchange anions, and on the other hand a polymer which is acidic in comparison to the basic polymer and which can exchange cations. The basic polymer has basic groups and is typically obtained by polymerisation of monomers, which carry basic groups, or groups, which can be converted into basic groups. These polymers are above all those, which have primary, secondary or tertiary amines, or the corresponding phosphines or at least two of the above functional groups. To this group of monomers belong in particular ethyleneamine, allylamine, diallylamine, 4-aminobutene, alkyloxyclycline, vinylformamide, 5-amino pentene, carbodiimide, formaldacine, melanine and the like, as well as secondary or tertiary amine derivatives thereof.

Preferred monoethylenically unsaturated, acidic group-containing monomers (α1) are acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbinic acid, α-chlorosorbinic acid, 2′-methylisocrotonic acid, cinnamic acid, p-chlorocinnamic acid, β-stearic acid, itaconic acid, citraconic acid, mesaconic acid, glutaconic acid, aconitic acid, maleic acid, fumaric acid, tricarboxythylene and maleic acid anhydride, where acrylic acid and methacrylic acid are particularly preferred and acrylic acid is even more particularly preferred.

Besides these carboxylate group-containing monomers, further preferred monoethylenically unsaturated acidic group-containing monomers (α1) are ethylenically unsaturated sulfonic acid monomers or ethylenically unsaturated phosphonic acid monomers.

Preferred ethylenically unsaturated sulfonic acid monomers are allylsulfonic acid or aliphatic or aromatic vinylsulfonic acids or acrylic or methacrylic sulfonic acids. Preferred aliphatic or aromatic vinylsulfonic acids are vinylsulfonic acid, 4-vinylbenzylsulfonic acid, vinyltoluenesulfonic acid and styrenesulfonic acid. Preferred acrylic or methylacrylic sulfonic acids are sulfoethyl(meth)acrylate, sulfopropyl(meth)acrylate and 2-hydroxy-3-methacryloxypropylsulfonic acid. As (meth)acrylamidoalkylsulfonic acid, 2-acrylamido-2-methylpropanesulfonic acid is preferred.

Additionally preferred are ethylenically unsaturated phosphonic acid monomers, such as vinylphosphonic acid, allylphosphonic acid, vinylbenzylphosphonic acid, (meth)acrylamidoalkylphosphonic acids, acrylamidoalkyldiphosphonic acids, phosphonomethylated vinylamines and (meth)acrylicphosphonic acid derivatives.

Preferred ethylenically unsaturated monomers (α1) comprising a protonated nitrogen are preferentially dialkylaminoethyl(meth)acrylates in the protonated form, for example dimethylaminoethyl(meth)acrylate-hydrochloride or dimethylaminoethyl(meth)acrylate-hydrosulfate, as well as dialkylaminoalkyl(meth)acrylamides in the protonated form, for example dimethylaminoethyl(meth)acrylamide-hydrochloride, dimethylaminopropyl(meth)acrylamide-hydrochloride, dimethylaminopropyl(meth)acrylamide-hydrosulfate or dimethylaminoethyl(meth)acrylamide-hydrosulfate.

Preferred ethylenically unsaturated monomers (α1) containing a quaternated nitrogen are dialkylammoniumalkyl(meth)acrylates in quaternated form, for example trimethylammoniumethyl(meth)acrylate-methosulfate or dimethylethylammoniumethyl(meth)acrylate-ethosulfate as well as (meth)acrylamidoalkyldialkylamines in quaternated form, for example (meth)acrylamidopropyltrimethylammonium chloride, trimethylammoniumethyl(meth)acrylate chloride and (meth)acrylamidopropyltrimethylammonium sulfate.

According to the invention it is preferred that the polymer comprises at least about 50 wt. %, preferably at least about 70 wt. % and more preferably at least about 90 wt. % carboxylate group-containing monomers. According to the invention it is particularly preferred that the polymer comprises at least about 50 wt. %, preferably at least about 70 wt. % acrylic acid, which is neutralised preferably to at least about 20 mol % and particularly preferably to at least about 50 mol %.

As monoethylenically unsaturated monomers (a2) co-polymerisable with (α1), acrylamides and (meth)acrylamides are preferred.

Possible (meth)acrylamides besides acrylamide and methacrylamide are alkyl-substituted (meth)acrylamides or aminoalkyl-substituted derivatives of (meth)acrylamides such as N-methylol(meth)acrylamide, N,N-dimethylamino(meth)acrylamide, dimethyl(meth)acrylamide or diethyl(meth)acrylamide. Possible vinylamides are for example N-vinylamides, N-vinylformamides, N-vinylacetamides, N-vinyl-N-methylacetamide, N-vinyl-N-methylformamides, vinylpyrrolidone. Among these monomers, acrylamide is particularly preferred.

Further preferred as monoethylenically unsaturated monomers (a2) which are copolymerisable with (α1) are water-dispersible monomers. Preferred as water-dispersible monomers are acrylic acid esters and methycrylic acid esters, such as methyl(meth)acrylate, ethyl(meth)acrylate, propyl(meth)acrylate or butyl(meth)acrylate, as well as vinylacetate, styrene and isobutylene. These water-dispersible monomers are preferably used in a quantity in the range from about 0.01 to about 20 wt %, preferably from about 0.1 to about 15 wt % and particularly preferably from about 0.5 to about 5 wt. %, based on the total amount of monomer used.

In addition, it is preferred according to the invention that the absorbent polymer is cross-linked by means of a chemical cross-linker (α3) or by thermal cross-linking or radiation cross-linking or at least two there from, wherein cross-linking by means of a chemical cross-linker (α3) is preferred.

Preferred cross-linkers (α3) according to the invention are compounds which have at least two ethylenically unsaturated groups in one molecule (cross-linker class I), compounds which have at least two functional groups which can react with functional groups of the monomers (α1) or (α2), preferably in a condensation reaction (=condensation cross-linkers), in an addition reaction or in a ring-opening reaction (cross-linker class II), compounds which have at least one ethylenically unsaturated group and at least one functional group which can react with functional groups of the monomers (α1) or (α2) (cross-linker class III), or polyvalent metal cations (cross-linker class (IV). Thus with the compounds of cross-linker class I, a cross-linking of the polymer is achieved by radical polymerisation of the ethylenically unsaturated groups of the cross-linker molecules with the monoethylenically unsaturated monomers (α1) or (α2), while with the compounds of cross-linker class II and the polyvalent metal cations of cross-linker class IV, a cross-linking of the polymer is achieved respectively via reaction of the functional groups (cross-linker class II) or via electrostatic interaction of the polyvalent metal cation (cross-linker class IV) with the functional groups of the monomer (α1) or (α2). With compounds of cross-linker class III a cross-linking of the polymers is achieved correspondingly by radical polymerisation of the ethylenically unsaturated groups as well as by reaction between the functional groups of the cross-linkers and the functional groups of the monomers (α1) or (α2).

Preferred compounds of cross-linker class I are poly(meth)acrylic acid esters, which have been obtained for example by conversion of a polyol, such as for example ethylene glycol, propylene glycol, trimethylolpropane, 1,6-hexanediol, glycerin, pentaerythritol, polyethyleneglycol or polypropyleneglycol, of an aminoalcohol, a polyalkylenepolyamine, such as for example diethylenetriamine or triethylenetetraamine, or of an alkoxylated polyol with acrylic acid or methacrylic acid. Further preferred as compounds of cross-linker class I are polyvinyl compounds, poly(meth)allyl compounds, (meth)acrylic acid esters of a monovinyl compound or (meth)acrylic acid esters of a mono(meth)allyl compound, preferably of the mono(meth)allyl compounds of a polyol or of an aminoalcohol. In this context DE 195 43 366 and DE 195 43 368, which are incorporated herein by reference to such compounds.

As examples of compounds of cross-linker class I are named alkenyldi(meth)acrylates, for example ethyleneglycoldi(meth)acrylate, 1,3-propyleneglycoldi(meth)acrylate, 1,4-butyleneglycoldi(meth)acrylate, 1,3-butyleneglycoldi(meth)acrylate, 1,6-hexanedioldi(meth)acrylate, 1,10-decanedioldi(meth)acrylate, 1,12-dodecanedioldi(meth)acrylate, 1,18-octadecanedioldi(meth)acrylate, cyclopentanedioldi(meth)acrylate, neopentylglycoldi(meth)acrylate, methylenedi(meth)acrylate or pentaerythritoldi(meth)acrylate, alkenyldi(meth)acrylamides, for example N-methyldi(meth)acrylamide, N,N′-3-methylbutylidenebis(meth)acrylamide, N,N′-(1,2-dihydroxyethylene)bis(meth)acrylamide, N,N′-hexamethylenebis(meth)acrylamide or N,N′-methylenebis(meth)acrylamide, polyalkoxydi(meth)acrylates, for example diethyleneglycoldi(meth)acrylate, triethyleneglycoldi(meth)acrylate, tetraethyleneglycoldi(meth)acrylate, dipropyleneglycoldi(meth)acrylate, tripropyleneglycoldi(meth)acrylate or tetrapropyleneglycoldi(meth)acrylate, bisphenol-A-di(meth)acrylate, ethoxylated bisphenol-A-di(meth)acrylate, benzylidenedi(meth)acrylate, 1,3-di(meth)acryloyloxypropanol-2, hydroquinonedi(meth)acrylate, di(meth)acrylate esters of trimethylolpropane, which are preferably alkoxylated with 1 to 30 mol alkylene oxide per hydroxyl group, preferably ethoxylated, thioethyleneglycoldi(meth)acrylate, thiopropyleneglycoldi(meth)acrylate, thiopolyethyleneglycoldi(meth)acrylate, thiopolypropyleneglycoldi(meth)acrylate, divinyl ethers, for example 1,4-butanedioldivinylether, divinyl esters, for example divinyladipate, alkanedienes, for example butadiene or 1,6-hexadiene, divinylbenzene, di(meth)allyl compounds, for example di(meth)allylphthalate or di(meth)allylsuccinate, homo- and co-polymers of di(meth)allyldimethylammonium chloride and homo- and co-polymers of diethyl(meth)allylaminomethyl(meth)acrylateammonium chloride, vinyl(meth)acrylic compounds, for example vinyl(meth)acrylate, (meth)allyl(meth)acrylic compounds, for example (meth)allyl(meth)acrylate, (meth)allyl(meth)acrylate ethoxylated with 1 to 30 mol ethylene oxide per hydroxyl group, di(meth)allylesters of polycarbonic acids, for example di(meth)allylmaleate, di(meth)allylfumarate, di(meth)allylsuccinate or di(meth)allylterephthalate, compounds with 3 or more ethylenically unsaturated, radically polymerisable groups such as for example glycerine tri(meth)acrylate, (meth)acrylate esters of glycerins ethoxylated with preferably 1 to 30 mol ethylene oxide per hydroxyl group, trimethylolpropanetri(meth)acrylate, tri(meth)acrylate esters of trimethylolpropane which is alkoxylated preferably with 1 to 30 mol alkylene oxide per hydroxide group, preferably ethoxylated, trimethacrylamide, (meth)allylidenedi(meth)acrylate, 3-allyloxy-1,2-propanedioldi(meth)acrylate, tri(meth)allylcyanurate, tri(meth)allylisocyanurate, pentaerythritoltetraa(meth)acrylate, pentaerythritoltri(meth)acrylate, (meth)acrylic acid esters of pentaerythritol which is ethoxylated with preferably 1 to 30 mol ethylene oxide per hydroxyl group, tris(2-hydroxyethyl)isocyanuratetri(meth)acrylate, trivinyltrimellitate, tri(meth)allylamine, di(meth)allylalkylamines, for example di(meth)allylmethylamine, tri(meth)allylphosphate, tetra(meth)allylethylenediamine, poly(meth)allyl ester, tetra(meth)allyloxyethane or tetra(meth)allylammonium halides.

Preferred as compounds of cross-linker class II are compounds which have at least two functional groups which can react with the functional groups of the monomers (α1) or (α2), preferably with acidic groups of the monomers (α1), in a condensation reaction (condensation cross-linkers), in an addition reaction or in a ring opening reaction. Examples of these functional groups of the compounds of crosslinker class II are preferably alcoholic, amino, aldehyde, glycidic, isocyanate, and carbonate or epichloro functions.

As examples of compounds of cross-linker class II are mentioned polyols, for example ethyleneglycol, polyethyleneglycols such as diethyleneglycol, triethyleneglycol and tetraethyleneglycol, propyleneglycol, polypropyleneglycols such as dipropyleneglycol, tripropyleneglycol or tetrapropyleneglycol, 1,3-butanediol, 1,4-butanediol, 1,5-pentanediol, 2,4-pentanediol, 1,6-hexanediol, 2,5-hexanediol, glycerine, polyglycerin, trimethylolpropane, polyoxypropylene, oxyethyleneoxypropylene-block copolymer, sorbitan-fatty acid esters, polyoxyethylenesorbitan-fatty acid esters, pentaerythritol, polyvinylalcohol and sorbitol, aminoalcohols, for example ethanolamine, diethanolamine, triethanolamine or propanolamine, polyamine compounds, for example ethylenediamine, diethylenetriamine, triethylenetetraamine, tetraethylenepentaamine or pentaethylenehexaamine, polyglycidyl ether compounds such as ethyleneglycoldiglycidyl ether, polyethyleneglycoldiglycidyl ether, glycerinediglycidyl ether, glycerinepolyglycidyl ether, pentaerithritolpolyglycidyl ether, propyleneglycoldiglycidyl ether, polypropyleneglycoldiglycidyl ether, neopentylglycoldiglycidyl ether, hexanediolglycidyl ether, trimethylolpropanepolyglycidyl ether, sorbitolpolyglycidyl ether, phthalic acid diglycidyl ester, adipinic acid diglycidyl ether, 1,4-phenylenebis(2-oxazoline), glycidol, polyisocyanates, preferably diisocyanates such as 2,4-toluenediioscyanate and hexamethylenediisocyanate, polyaziridine compounds such as 2,2-bishydroxymethylbutanol-tris[3-(1-aziridinyl)propionate], 1,6-hexamethylenediethyleneurea and diphenylmethane-bis-4,4′-N,N′-diethyleneurea, halogen epoxides for example epichloro- and epibromohydrin and α-methylepichlorohydrin, alkylenecarbonates such as 1,3-dioxolane-2-one (ethylene carbonate), 4-methyl-1,3-dioxolane-2-one (propylene carbonate), 4,5-dimethyl-1,3-dioxolane-2-one, 4,4-dimethyl-1,3-dioxolane-2-one, 4-ethyl-1,3-dioxolane-2-one, 4-hydroxymethyl-1,1-dioxolane-2-one, 1,3-dioxane-2-one, 4-methyl-1,3-dioxane-2-one, 4,6-dimethyl-1,3-dioxane-2-one, 1,3-dioxolane-2-one, poly-1,3-dioxolane-2-on, polyquaternary amines such as condensation products from dimethylamines and epichlorohydrin. Further preferred compounds of the crosslinker class II are in addition polyoxazolines such as 1,2-ethylenebisoxazoline, cross-linkers with silane groups such as γ-glycidooxypropyltrimethoxysilane and γ-aminopropyltrimethoxysilane, oxazolidinones such as 2-oxazolidinone, bis- and poly-2-oxazolidinone and diglycolsilicates.

Preferred as compounds of class III are hydroxyl or amino group-containing esters of (meth)acrylic acid, such as for example 2-hydroxyethyl(meth)acrylate, as well as hydroxyl or amino group-containing (meth)acrylamides, or mono(meth)allylic compounds of diols.

The polyvalent metal cations of the cross-linker class IV are derived preferably from singly or multiply charged cations, the singly charged in particular from alkali metals such as potassium, sodium, lithium, wherein lithium is preferred. Preferred doubly charged cations are derived from zinc, beryllium, alkaline earth metals such as magnesium, calcium, strontium, wherein magnesium is preferred. Further applicable cations with higher charge are cations from aluminium, iron, chromium, manganese, titanium, zirconium and other transition metals as well as double salts of such cations or mixtures of the named salts. Preferably used are aluminium compounds such as the poly-aluminium compounds obtainable under the name PaperPAC-N or aluminium salts and alums and various hydrates thereof such as e.g. AlCl₃×6H₂O, NaAl(SO₄)₂×12H₂O, KAl(SO₄)₂×12H₂O or Al₂(SO₄)₃×14-18H₂O.

The use of Al₂(SO₄)₃ and its hydrates as cross-linkers of cross-linker class IV is particularly preferred.

Preferred absorbent polymers are polymers which are cross-linked by crosslinkers of the following cross-linker classes or by cross-linkers of the following combinations of cross-linker classes: I, II, III, IV, I II, I III, I IV, I II III, I II IV, I III IV, II III IV, II IV or III IV. The above combinations of cross-linker classes represent respectively a preferred embodiment of cross-linkers of an absorbent polymer.

Further preferred embodiments of the absorbent polymers are polymers which are cross-linked by any of the above named cross-linkers of cross-linker class I. Among these, water soluble cross-linkers are preferred. In this context, N,N′-methylenebisacrylamide, polyethyleneglycoldi(meth)acrylate, triallylmethylammonium chloride, tetraallylammonium chloride as well as allylnonaethyleneglycolacrylate made with 9 mol ethylene oxide per mol acrylic acid are particularly preferred.

The absorbent polymer can be produced from the above-named monomers and cross-linkers by various polymerisation means. For example, in this context can be named bulk polymerisation, solution polymerisation, spray polymerisation, inverse emulsion polymerisation and inverse suspension polymerisation. Solution polymerisation is preferably carried out in water as solvent. The solution polymerisation can occur continuously or discontinuously. From the prior art a broad spectrum of variation possibilities can be gathered with respect to reaction proportions such as temperature, type and quantity of the initiators as well as of the reaction solution. Typical processes are described in the following patent specifications: U.S. Pat. No. 4,286,082, DE 27 06 135, U.S. Pat. No. 4,076,663, DE 35 03 458, DE 40 20 780, DE 42 44 548, DE 43 23 001, DE 43 33 056, DE 44 18 818 which are incorpotated by reference as to typical processes disclosed therein.

Another possibility for producing the absorbent polymers is to first produce non-cross-linked, in particular linear polymers, preferably by radical means from the above named monoethylenically unsaturated monomers (α1) or (α2) and then to convert these with reagents acting as a cross-linker (α3), preferably with those of classes II and IV. This variant is then used preferentially if the polymer structure should be first processed in form-giving processes, for example to form fibres, films or other flat structures such as fabrics, knitted fabrics, webs or fleeces, and cross-linked in this form.

Polymerisation initiators can be dissolved or dispersed in a solution of monomer according to the invention. All compounds decomposing to form radicals under the polymerisation conditions and known to the skilled person are considered as initiators. Hereunder fall in particular peroxides, hydroperoxides, hydrogen peroxide, persulfates, azo compounds as well as the so-called redox catalysts. Preferred is the use of water soluble catalysts. In some cases it is advantageous to use mixtures of different polymerisation initiators. Among these mixtures are preferred those comprising hydrogen peroxide and sodium or potassium peroxodisulfate, which can be used in any conceivable quantity ratio. Suitable organic peroxides are preferably acetylacetone peroxide, methylethylketone peroxide, tbutylhydroperoxide, cumolhydroperoxide, t-amylperpivalate, t-butylperpivalate, tbutylperneohexonate, t-butylisobutyrate, t-butylper-2-ethylhexenoate, tbutylperisononanoate, t-butylpermaleate, t-butylperbenzoate, t-butyl-3,5,5-trimethylhexanoate and amylperneodecanoate. Additionally preferred as polymerisation initiators are: azo compounds, such as 2,2′-azobis(2-amidinopropane)dihydrochloride, azo-bisamidinopropane-dihydrochloride, 2,2′-azobis(N,N-dimethylene)isobutyramidine-dihydrochloride, 2-(carbamoylazo)isobutyronitrile and 4,4′-azobis(4-cyanovaleric acid). The compounds mentioned are used in normal quantities, preferably within a range from about 0.01 to about 5, preferably from about 0.1 to about 2 mol %, respectively based on the quantity of the monomers to be polymerised.

The redox catalysts have as oxidic components at least one of the above-indicated per-compounds and as reducing components preferably ascorbic acid, glucose, sorbose, mannose, ammonium or alkali metal hydrogensulfite, -sulfate, -thiosulfate, -hyposulfite or -sulfide, metal salts, such as iron(II) ions or silver ions or sodium hydroxymethylsulfoxylate. Preferably used as reducing components of the redox catalysts are ascorbic acid or sodium pyrosulfite. Based oh the quantity of monomers used in the polymerisation, from about 1×10⁻⁵ to about 1 mol % of the reducing component of the redox catalyst and from about 1×10⁻⁵ to about 5 mol % of the oxidising component of the redox catalyst are used. In place of the oxidising components of the redox catalyst, or in complement thereto, one or more preferably water soluble azo compounds can be used.

A redox system used preferentially according to the invention comprises hydrogen peroxide, sodium peroxodisulfate and ascorbic acid. Generally azo compounds are preferred as initiators according to the invention, wherein azobis(amidinopropane) dihydrochloride is particularly preferred. As a rule the polymerisation is initiated with the initiators in a temperature range of from about 30 to about 90° C.

As so-called “post cross-linker”, with which the absorbent polymer is treated preferably additionally mostly in the area of the surface of the polymer particles, compounds mentioned in the context of the cross-linkers (α3) of cross-linker classes II and IV are particularly suitable.

Among these compounds, as post cross-linker are particularly preferred diethylene glycol, triethylene glycol, polyethylene glycol, glycerine, polyglycerine, propylene glycol, diethanolamine, triethanolamine, polyoxypropylene, oxyethyleneoxypropylene block copolymers, sorbitan fatty acid esters, polyoxyethylenesorbitan fatty acid esters, trimethylolpropane, pentaerythritol, polyvinylalcohol, sorbitol, 1,3-dioxolane-2-one (ethylene carbonate), 4-methyl-1,3-dioxolane-2-one (propylene carbonate), 4,5-dimethyl-1,3-dioxolane-2-one, 4,4-dimethyl-1,3-dioxolane-2-one, 4-ethyl-1,3-dioxolane-2-one, 4-hydroxymethyl-1,3-dioxolane-2-one, 1,3-dioxane-2-one, 4-methyl-1,3-dioxane-2-one, 4,6-dimethyl-1,3-dioxane-2-one, 1,3-dioxolane-2-one, poly-1,3-dioxolane-2-one. Particularly preferably, ethylene carbonate is used as post cross-linker.

Preferred embodiments of the polymers are those, which are post-cross-linked by means of cross-linkers of the following cross-linker classes or respectively by means of cross-linkers of the following combinations of cross-linker classes: II, IV and II IV.

Further preferred embodiments of the polymers are those, which are post-crosslinked by means of any of the cross-linkers mentioned above in the cross-linker classes II or IV.

These compounds are preferably used in a quantity in the range from about 0.01 to about 30 wt %, preferably from about 0.1 to about 20 wt % and particularly preferably from 0.5 to about 10 wt. %, based on the thus far untreated polymer. Organic solvents can be added to the mixture in a quantity from 0 to about 60 wt %, preferably from 0.1 to about 40 wt % and particularly preferably from 0.2 to about 50 wt. %, based on the thus far untreated polymer. As organic solvent are preferred lower alcohols such as methanol, ethanol, n-propanol, isopropanol, n-butanol, isobutanol, sec-butanol and t-butanol, ketones such as acetone, methylethylketone and methylisobutylketone, ethers such as dioxane, tetrahydrofuran and diethylether, amides such as N,N-dimethylformamide and N,N-diethylformamide as well as sulfoxides such as dimethylsulfoxide.

As water soluble polymers (α4), water soluble polymerisates such as those comprising partly or fully saponified polyvinyl alcohol, polyvinylpyrrolidone, starches or starch derivatives, polyglycols or polyacrylic acids can preferably be polymerised into the absorbent polymers. The molecular weight of these polymers is not critical, as long as they are water soluble. Preferred water soluble polymers are starches, starch derivatives, or polyvinyl alcohol. The water soluble polymers, preferably synthetic like polyvinyl alcohol, can also serve as graft basis for the monomers to be polymerised.

As auxiliaries (α5), preferably suspension agents, surface-active agents, odour binders or antioxidants are included in the absorbent polymers. These auxiliaries are preferably added to the monomer solution.

It is further preferred according to the invention that the absorbent polymers have an inner portion, an outer portion surrounding the inner portion and a surface portion surrounding the outer portion, whereby the outer portion has a higher degree of cross-linking than the inner portion, so that preferably a core-shell structure forms. The increased cross-linking in the surface portion of the polymer is thus achieved preferably by post-cross-linking of surface-near, reactive groups. This post-cross-linking can occur thermally, photochemically or chemically. As post-cross-linker for the chemical post-cross-linking are thus preferred compounds which were mentioned as cross-linkers (α3) of cross-linker classes II and IV. Particularly preferred as post-cross-linker is ethylene carbonate.

Preferably, the absorbent material and in particular the absorbent polymer has at least one of the following properties:
(a1) maximum absorption of 0.9 wt. % aqueous NaCl solution according to ERT 440.1-99 in the range from at least about 10 to about 1000, preferably from about 15 to about 500 and particularly preferably from about 20 to about 300 ml/g,
(b1) the part extractable with 0.9 wt. % aqueous NaCl solution according to ERT 470.1-99 amounts to less than about 30 wt %, preferably less than about 20 wt % and particularly preferably less than 10 wt. %, based on the untreated absorbent polymer structure,
(c1) the swelling time to reach about 80% of the maximum absorption of 0.9 wt. % aqueous NaCl solution according to ERT 440.1-99 lies in the range from about 0.01 to about 180 min, preferably from about 0.01 to about 150 min and particularly preferably from about 0.01 to about 100 min.,
(d1) the bulk density according to ERT 460.1-99 lies in the range from about 300 to about 1000, preferably from about 310 to about 800 and particularly preferably from about 320 to about 700 g/l,
(e1) the pH value according to ERT 400.1-99 for 1 g of the untreated absorbent polymer structure in 1 l water lies in the range from about 4 to about 10, preferably from about 5 to about 9 and particularly preferably from about 5.5 to about 7.5.
(f1) CRC according to ERT 441.1-99 in the range from about 10 to about 100 g/g, preferably from about 15 to about 80 g/g and particularly preferably from about 20 to about 60 g/g.
(g1) AAP according to ERT 442.1-99 in the range from about 10 to about 60, preferably from about 15 to about 50 and particularly preferably from about 20 to about 40 g/g.

The property combinations of two or more properties arising from the above properties represent respectively, in addition to each individual property, preferred embodiments according to the invention. Further particularly preferred as embodiments according to the invention are the following properties or combinations of properties depicted as alphabetic characters or combinations of alphabetic characters: a1, b1, c1, d1, e1, f1, g1, a1b1, a1c1, a1d1, a1f1, a1g1, f1b1, f1c1, f1d1, g1b1, g1c1, g1d1, f1g1c1, f1g1d1, f1g1b1, a1f1b1, a1f1c1, a1g1b1, a1g1c1, a1f1d1, a1g1d1, a1f1g1, a1f1g1b1, a1f1g1c1, a1f1g1d1, a1f1b1c1, a1g1b1c1, a1f1d1c1, a1g1d1b1, a1f1b1d1, a1g1c1d1, f1g1c1b1, f1g1d1b1, f1g1c1d1, a1f1g1b1c1, a1f1g1c1d1, a1f1g1d1b1, a1b1c1d1f1g1, a1b1c1d1e1f1g1.

The invention further relates to a process for producing an absorbent hygiene article, wherein (a) a stiff element which stiffens on contact with body fluids or (b) a gas element which forms a gas on contact with fluids or both are worked into (c) at least one hygiene article component.

In the process according to the invention, it is preferred, that the incorporation occurs by mixing in the stiff element of the gas element, which forms a gas. In another embodiment the stiff element or the gas element, which forms a gas, can be laid between different hygiene article component layers in the hygiene article production. This can occur, for example, in a diaper machine, in which the stiff element or the gas element, which forms a gas, are fabricated as cases introduced between or on the corresponding hygiene component layer or layers.

The invention further relates to an absorbent hygiene article, which is obtainable by means of the above described process according to the invention.

In addition, the invention relates to the use of the absorbent hygiene article according to the invention and of the absorbent hygiene article obtainable by means of the process according to the invention for supporting the becoming dry of children in the age range of 0.5 to 5 years.

The invention additionally relates to the use of a (α) stiff element which stiffens on contact with body fluids or a (β) gas element which forms a gas on contact with body fluids or both, in a hygiene article for reducing the tendency of wetting themselves of a human wearer or as a wetting indicator. As stiff element and as gas element which forms a gas are thus preferred all elements which have already been mentioned in the context of the absorbent hygiene article according to the invention.

The invention is now more closely illustrated by means of non-limiting diagrams and examples.

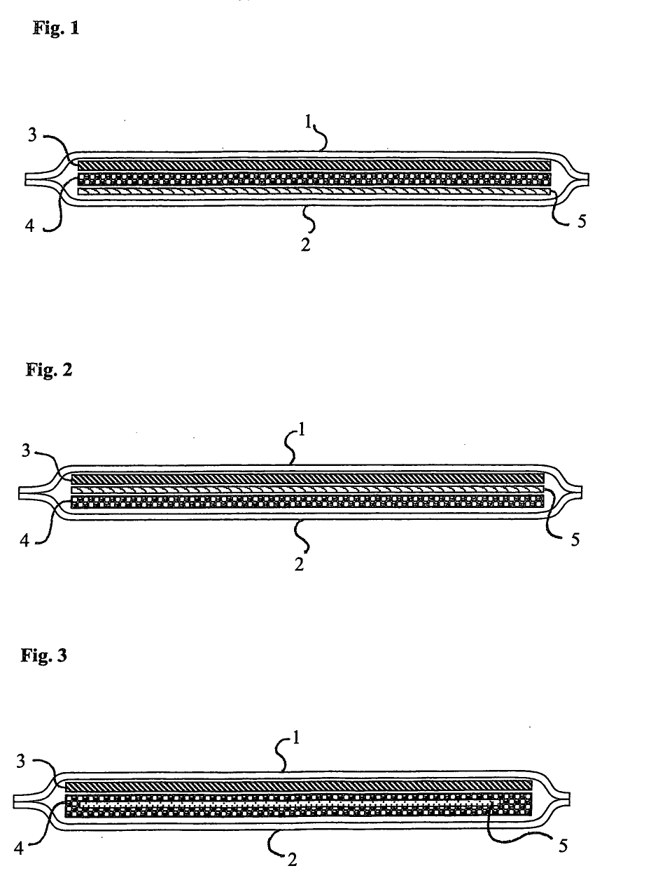

In FIG. 1 a construction of a hygiene article is shown, in which is arranged, between a liquid permeable cover layer 1 and an outer layer 2, a distribution layer 3 adjacent to the cover layer 1, followed by an absorption layer 4 and a layer comprising a stiff element or a gas element which forms a gas or both, wherein a surface of the layer 5 comprising a stiff element and a gas element which forms a gas faces the outer layer 2.

FIG. 2 shows a construction of a further embodiment of the hygiene article according to the invention, wherein in contrary to FIG. 1, the layer 5 comprising the stiff element or the gas element which forms a gas or both is arranged between the distribution layer 3 and the absorption layer 4.

FIG. 3 shows the construction of a hygiene article which differs from the construction according to FIG. 1 in that the layer 5 comprising the stiff element or the gas element which forms a gas or both is incorporated into the absorption layer 4, wherein the layer 5 is also surrounded by the absorption layer 4 on the side of the construction in which the cover layer 1 and the outer layer 2 adjoin each other.

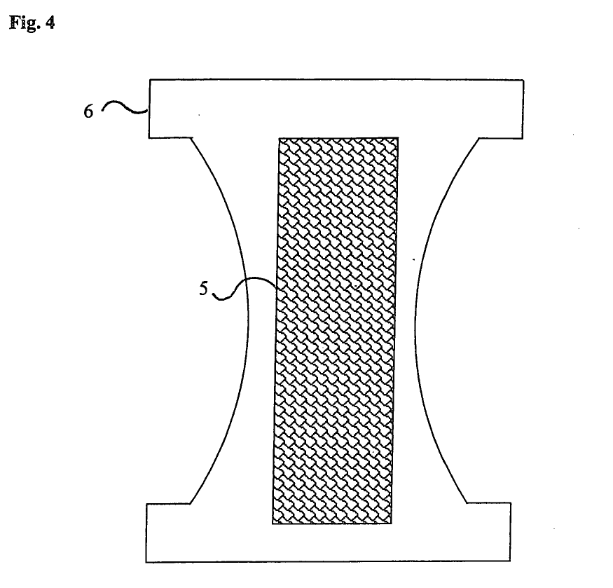

FIG. 4 shows the view of a hygiene article 6, in which a layer 5 comprising a stiff element or a gas element which forms a gas is arranged, wherein the limits of layer 5 lie within the limits of hygiene article 6 and the longest dimension of layer 5 and the longest dimension of hygiene article 6 correspond and the area of layer 5 with respect to hygiene article 6 covers between one third and two thirds of the area of hygiene article 6.

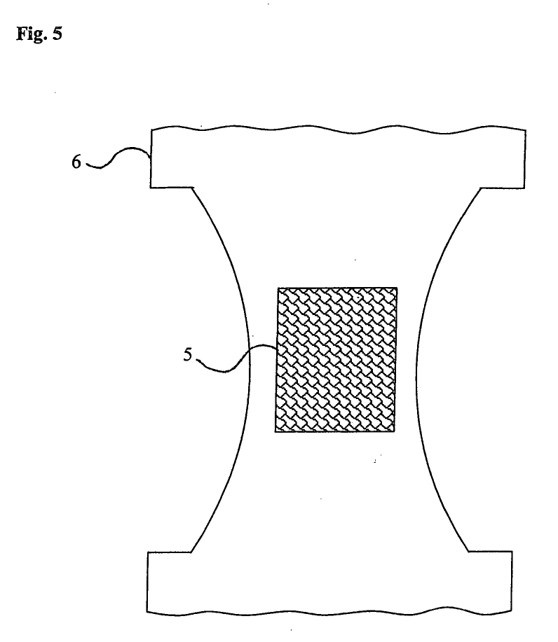

FIG. 5 shows a hygiene article, which differs from the hygiene article of FIG. 4 in that the area of layer 5 in comparison to hygiene article 6 represents between one fifth and one third of the area of hygiene article 6.

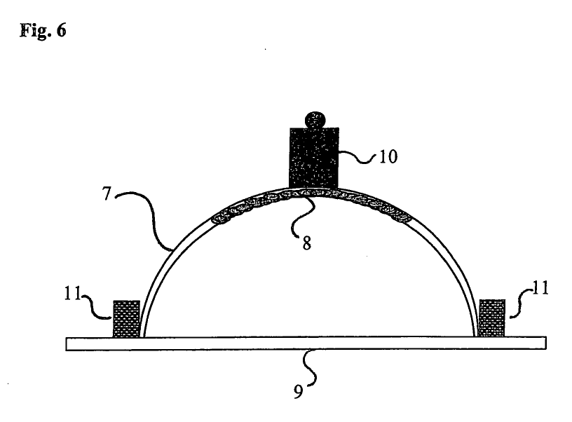

FIG. 6 displays the test methods for force measurement.

Test Methods

Determination of the Stiffness of a Diaper with Stiff Element

For the stiffness of a diaper, a diaper (7), comprising a stiff element (8) and removed from the body-shaped apparatus (kanga), which had been brought into contact with fluid, was removed and with both ends placed upon a flat support (9) (see FIG. 6), so that it forms a half ring. A sideways slipping away of the diaper is prevented by means of two lateral restraints (11). In the middle of the highest position of the diaper, weights (10) of 500 g, 1000 g and 1500 g are placed one after the other. The weight at which the middle of the diaper contacts the support within 30 seconds is tested. Diapers without the stiff element cannot be stably placed with both hands on a support. They collapse immediately.

Determination of Gas Evolution in a Diaper with Gas Element

The diaper with pad is spread out flat on a table and fixed. A plastic plate with the dimensions of the pad is laid at the position where the pad lies on the surface of the diaper. The weight of the plate should be selected so that a force of 2 g/cm² acts on the diaper surface. A graduated measuring rod is placed besides the plate. The plate contains a feed opening in the form of a tube, into which a corresponding quantity of fluid is added. It is then observed, when the plate rises by at least 2 mm and how long this lasts.

### EXAMPLES

1. A diaper of the brand HUGGIES® (a registered trademark of Kimberly-Clark Worldwide Inc., and manufacturer Kimberly-Clark, Barton, GB) was split at the side. A layer of a fast-setting plaster bandage of the brand PLASTRONA® a registered trademark of the company Paul Hartmann AG, was placed between the absorption layer and the outer layer (see FIG. 1 and FIG. 4), so that it was positioned in the middle in the longitudinal direction of the diaper. The diaper was clamped in a body-shaped apparatus (Kanga) and wet with 200 ml of 0.9% NaCl solution. After approx. 10 minutes, the diaper was removed and placed the other way up on a flat support. The lower side of the diaper could not be forced onto the flat support by laying on a weight of 500 g. With 1000 g the underside of the diaper was forced onto the support within 30 seconds.

2. A diaper of the brand HUGGIES® (manufacturer Kimberly-Clark, Barton, GB) was split at the side. A layer of an artificial plaster bandage of the brand SCOTCHCAST®, a registered trademark of 3M Corporation, and of the company Laboratories 3M Santé was placed between the absorption layer and the outer layer (see FIG. 1 and FIG. 4), so that it was positioned in the middle of the longitudinal direction of the diaper. The diaper was clamped in a body-shaped apparatus (Kanga) and wet with 200 ml of 0.9% NaCl solution. After approximately 10 minutes the diaper was removed and placed the other way up on a flat support. The underside of the diaper could not be forced onto the flat support by means of laying on a weight of 500 g. With 1000 g the underside of the diaper was forced onto the support within 30 seconds.

3. From two sheets with a length of 10 cm and a width of 8 cm of a heat sealable material (Dexter Paper, non-woven: mass per area: 16.5±1.5 g/m², thermoplastic fibre content: 4±0.8 g/m², wet tensile strength in transverse direction: 70±12 N/m, air permeability: 230±50 L/min/100 cm²) a pouch was sealed, into which 10 g of a mixture of 0.5 of superabsorber FAVOR® SXM 880 of the company Stockhausen GmbH & Co. Kg and 9.5 g of plaster from the company Knauf Bauprodukte (Postal Box 10, 97343 Iphofen) had been inserted. The pouch was then sealed closed and at the same time provided with further sealing points, which were distributed over the surface of the pouch, to prevent shifting of the powder. The pouch was placed between absorption and outer layer of a diaper of the brand HUGGIES® (manufacturer Kimberly-Clark, Barton, GB) according to FIG. 5. The diaper was clamped in a body-shaped apparatus (Kanga) and wet with 200 ml of 0.9% NaCl solution. After approximately 10 minutes the diaper was removed and placed upside down on a flat support. The underside of the diaper could not be forced onto the flat support by laying on a weight of 500 g. With 1000 g the underside of the diaper was forced onto the support within 30 seconds.

4. From two sheets with a length of 10 cm and a width of 8 cm of a heat-sealable material (Dexter Paper, non-woven: mass per surface: 16.5±1.5 g/m², thermoplastic fibre content: 4±0.8 g/m², wet tensile strength in transverse direction: 70±12 N/m, air permeability: 230±50 L/min/100 cm²) a pouch was sealed, into which 10 g of a mixture of 2.0 g superabsorber FAVOR® SXM 880 of the company Stockhausen GmbH & Co. Kg, 3 g citric acid and 4 g sodium hydrogencarbonate were introduced. The pouch was then sealed closed and at the same time provided with further sealing points, which were distributed over the surface of the pouch, to prevent a shifting of the powder. The pouch was placed between absorption and distribution layer of a diaper of the brand HUGGIES® (manufacturer Kimberly-Clark, Barton, GB) corresponding to FIG. 5. The diaper was tested using the above-described test methods in respect of gas evolution. After addition of 100 ml of a 0.9 wt. % NaCl solution, the plate was raised to a height of 2 mm over a time of 10 minutes.

5. From two sheets with a length of 20 cm and a width of 4 cm of a heatsealable material (Dexter Paper, non-woven: mass per surface: 16.5±1.5 g/m², thermoplastic fibre content: 4±0.8 g/m², wet tensile strength in transverse direction: 70±12 N/m, air permeability: 230±50 L/min/100 cm²) a pouch was sealed, into which 16 g of a mixture of 4.0 g superabsorber FAVOR® SXM 880 of the company Stockhausen GmbH & Co. Kg, 5 g citric acid and 7 g sodium hydrogencarbonate were introduced. The pouch was then sealed closed. Moreover, in doing so, provided with further sealing points, which were distributed over the surface of the pouch, in order to prevent a shifting of the powder. The pouch was placed between absorption and distribution layer of a diaper of the brand HUGGIES® (manufacturer Kimberly-Clark, Barton, GB) corresponding to FIG. 4. The diaper was tested using the above-described test methods in respect of gas evolution. After addition of 150 ml of a 0.9 wt. % and NaCl solution, the plate was raised to a height of 2 mm over a time of 16 minutes.

1. An absorbent hygiene article comprising:
(a) an indicator element selected from the group consisting of a resin which stiffens on contact with body fluids, an inorganic compound which stiffens on contact with body fluids, and
a gas element which forms a gas on contact with body fluids wherein said gas can force out; and
(b) a hygiene article component,
wherein the indicator element is in contact with the hygiene article component.

2. The absorbent hygiene article of claim 1, wherein the gas is CO₂ or N₂.

3. The absorbent hygiene article of claim 1, wherein the indicator element is arranged in a sheath.

4. The absorbent hygiene article of claim 1, wherein said hygiene article component comprises:
(A) a cover layer which is at least partially permeable to body fluids,
(B) optionally an outer layer,
(C) optionally a distribution layer,
(D) an absorption layer, and
(E) a layer comprising the indicator element,
wherein the cover layer, optionally the distribution layer as well as the absorption layer, if the article is brought into contact with a liquid, are at least partially in contact with the liquid and are optionally at least partially surrounded by the outer layer, wherein the layer comprising the indicator element is at least partially arranged according to a variant
(i) between the absorption layer and the outer layer, or
(ii) between the distribution layer and the absorption layer, or
(iii) in the absorption layer.

5. The absorbent hygiene article of claim 4, wherein the absorption layer comprises an absorbent polymer.

6. A process for producing an absorbent hygiene article, comprising the step of combining:
(a) an indicator element selected from the group consisting of a resin which stiffens on contact with body fluids, an inorganic compound which stiffens on contact with body fluids, and
a gas element which forms a gas on contact with body fluids wherein said gas can force out; and
(b) at least one hygiene article component.

7. Use of an absorbent hygiene article according to any one of claims 1 to 5 for supporting the becoming dry of children in the age range from 0.5 to 5 years.

8. Use of a
(a) resin which stiffens on contact with body fluids or an inorganic compound which stiffens on contact with body fluids as stiff element, or
(b) gas element which forms a gas on contact with body fluids and out of which the gas formed can force out, or both
in a hygiene article for reducing the tendency of a human wearer to wet themself or
as a wetting themself indicator.
